(19) United States
(12) Patent Application Publication
(10) Pub. No.: US 2026/0354702 A1
(43) Pub. Date: Nov. 12, 2026

(54) HUMAN-BODY PRESSURE SENSING SYSTEM

(71) Applicant: DotSpace Inc., County of New Castle, DE (US)

(72) Inventors: UEIMING JOW, County of New Castle, DE (US); YEN LIANG LAI, Taipei City (TW)

(21) Appl. No.: 19/122,977

(22) PCT Filed: Nov. 6, 2023
(86) PCT No.: PCT/US2023/036856

§ 371 Date: Apr. 21, 2025

Related U.S. Application Data
(60) Provisional application No. 63/426,201, filed on Nov. 17, 2022.

(51) Int. Cl. A61B 5/03; A61B 5/00; A61B 5/01

(52) U.S. Cl. CPC A61B 5/036; A61B 5/01; A61B 5/4255; A61B 5/742; A61B 5/746

(57) ABSTRACT

A human-body pressure sensing system includes a human-body information sensing device and an off-body processing device. The human-body information sensing device has a first wireless communication unit, a first pressure sensing unit, and an activating unit. The first pressure sensing unit senses an intra-body absolute pressure value inside a human body, and outputs the intra-body absolute pressure value using the first wireless communication unit. The off-body processing device has a second wireless communication unit, a second pressure sensing unit, and a pressure calculating unit. The second wireless communication unit is in communication with the first wireless communication unit. The second pressure sensing unit senses an ambient absolute pressure value. The pressure calculating unit receives the intra-body absolute pressure value and the ambient absolute pressure value, and determines an intra-body relative pressure value by calculating a difference between the intra-body absolute pressure value and the ambient absolute pressure value.

### BACKGROUND OF THE INVENTION

### 1. Technical Field

The present invention relates to sensing system, and more particularly to a human-body pressure sensing system.

### 2. Description of Related Art

In the medial domain, sensors are extensively used to collect and monitor various vital signs of patients so as to facilitate timely responsive actions. For example, different pressure sensors may be used to detect intra-body pressures at different parts of a patient.

To this end, capsule pressure sensors have been recognized as an effective means. However, existing capsule pressure sensors are designed for collecting absolute pressures in a human body, and are less accurate when used to monitor vital signs that can only be obtained through relative pressures (i.e., the pressures in a human body with respective to the pressure of the ambient environment), such as the intra-abdominal pressure. Hence, there is a need for a pressure sensor capable of providing accurate relative pressures of a patient.

### SUMMARY OF THE INVENTION

In order to meet the aforementioned need, the present invention discloses a human-body pressure sensing system, which is able to accurately detect an intra-body absolute pressure value and an intra-body relative pressure value related to a human body of a user, so as to provide medical staff with accurate and diverse data and to achieve monitoring of vital signs of patients.

In order to achieve the foregoing objectives, an embodiment of the present invention provides a human-body pressure sensing system, which comprises a human-body information sensing device, having a first wireless communication unit, a first pressure sensing unit, and an activating unit, in which the first pressure sensing unit senses an intra-body absolute pressure value inside a human body of a user, and outputs the intra-body absolute pressure value through the first wireless communication unit, so as that the activating unit controls the human-body information sensing device to start operation; and an off-body processing device, having a second wireless communication unit, a second pressure sensing unit, and a pressure calculating unit, in which the second wireless communication unit is in communication with the first wireless communication unit, and the second pressure sensing unit senses an ambient absolute pressure value, so that the pressure calculating unit receives the intra-body absolute pressure value and the ambient absolute pressure value, and determines an intra-body relative pressure value by calculating a difference between the intra-body absolute pressure value and the ambient absolute pressure value.

Thereby, the human-body information sensing device and the off-body processing device of the present invention can accurately detect the intra-body absolute pressure value and the intra-body relative pressure value inside a human body, and provide medical staff with accurate and diverse data, thereby achieving competent monitoring of vital signs of patients.

### BRIEF DESCRIPTION OF THE DRAWINGS

FIG. 1 is a block diagram illustrating connection of a human-body pressure sensing system according to one embodiment of the present invention.

FIG. 2 is a block diagram illustrating connection of a human-body information sensing device according to one embodiment of the present invention.

FIG. 3 is a block diagram illustrating connection of an off-body processing device according to one embodiment of the present invention.

FIG. 4 is a perspective view of a human-body information sensing device of one embodiment of the present invention.

### DETAILED DESCRIPTION OF THE INVENTION

The following preferred embodiments when read with the accompanying drawings are made to clearly exhibit the above-mentioned and other technical contents, features, and effects of the present invention. Through the exposition by means of the specific embodiments, people would further understand the technical means and effects the present invention adopts to achieve the above-indicated objectives. However, the accompanying drawings are intended for reference and illustration, but not to limit the present invention and are not made to scale.

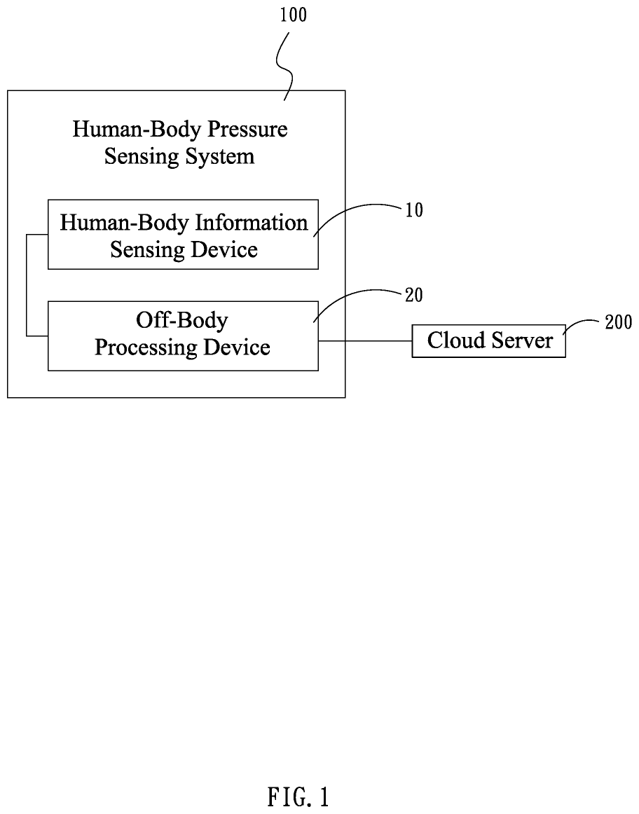

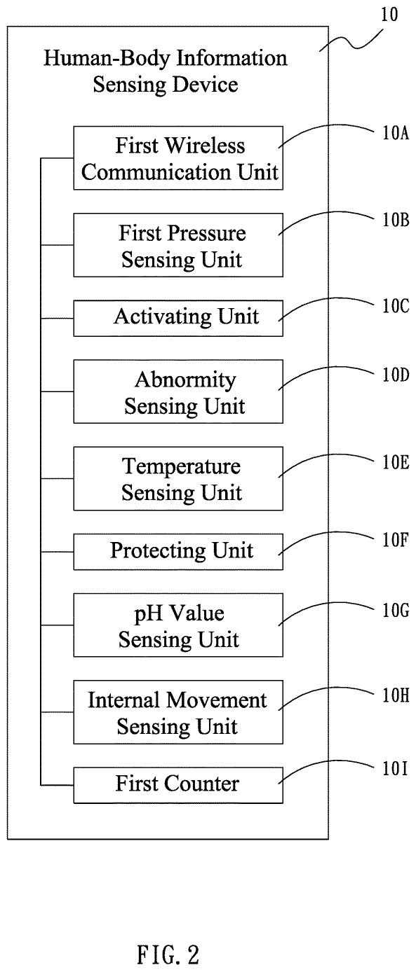

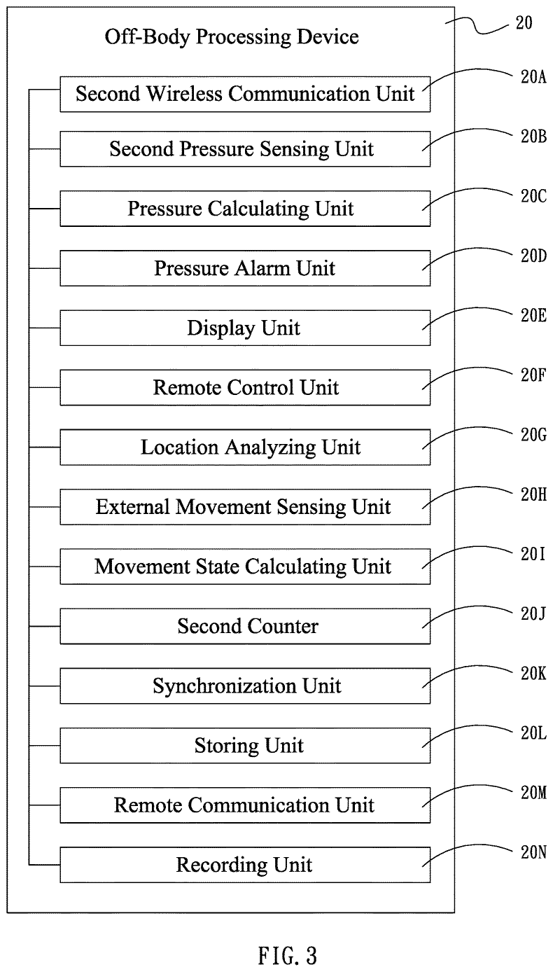

Referring to FIG. 1 through FIG. 4, in an embodiment of the present invention, a human-body pressure sensing system 100 comprises a human-body information sensing device 10 and an off-body processing device 20 that are in communication with each other. Therein, according to the embodiment of the present invention, the human-body information sensing device 10 is in the form of a capsule for a user to swallow and ingest into his/her body, so that the human-body information sensing device 10 can detect various data inside the body of the user, and the off-body processing device 20 may be a wearable device such as a chest band or a waist band equipped with a smart device.

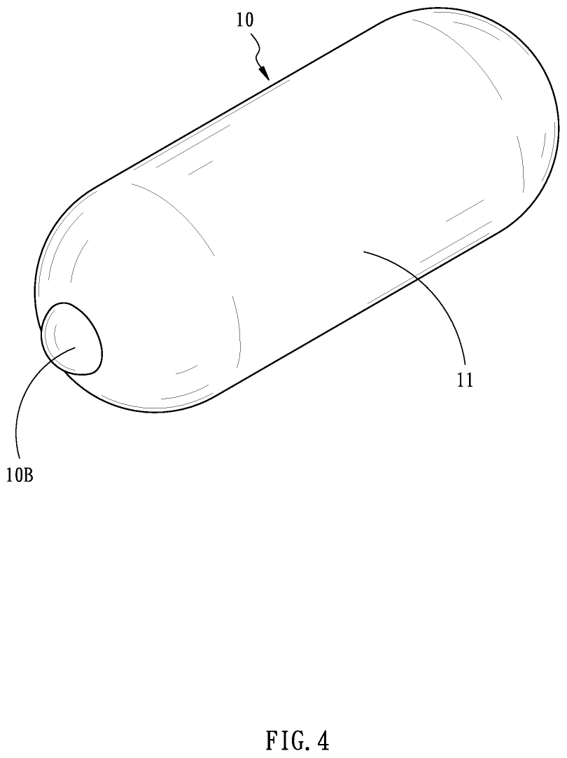

The human-body information sensing device 10 has a first wireless communication unit 10A, a first pressure sensing unit 10B, and an activating unit 10C that are coupled to each other. The first pressure sensing unit 10B senses an intra-body absolute pressure value inside the human body, and the first wireless communication unit 10A outputs the intra-body absolute pressure value to the off-body processing device 20. The activating unit 10C controls the human-body information sensing device 10 to start operation. Therein, as shown in FIG. 4, the human-body information sensing device 10 has a casing 11, and the first pressure sensing unit 10B is such installed in the casing 11 that it is partially exposed outside the casing 11, thereby maximizing measurement accuracy of the intra-body absolute pressure value.

Additionally, in the embodiment of the present invention, the activating unit 10C is a non-contact switch such as a magnetic switch or a microwave switch. In an example where the activating unit 10C is implemented using a magnetic switch, the user may make the activating unit 10C control the human-body information sensing device 10 to start to operate by drawing a magnetic object (e.g., a magnet) close to the activating unit 10C. In the example of the present invention, the magnetic object has a magnetic flux density higher than 3 millitesla (mT) in order to activate the activating unit 10C. In another example where the activating unit 10C is implemented using a microwave switch, the off-body processing device 20 is configured to emit a particular signal to the activating unit 10C to make the activating unit 10C control the human-body information sensing device 10 to start to operate. Since the activating unit 10C of the human-body information sensing device 10 is a non-contact switch, the user has the freedom to decide whether the human-body information sensing device 10 is to be activated outside or inside the human body, making use of the disclosed system more versatile and convenient.

As shown in FIG. 1 through FIG. 3, in the embodiment of the present invention, the human-body information sensing device 10 comprises an abnormity sensing unit 10D coupled to the first wireless communication unit 10A. The abnormity sensing unit 10D senses operation of the human-body information sensing device 10. When the abnormity sensing unit 10D detects abnormal operation, an operational abnormity signal is outputted to the off-body processing device 20 through the first wireless communication unit 10A. Then the off-body processing device 20, according to the operational abnormity signal, displays an operational abnormity message to inform the user of the abnormity of the human-body information sensing device 10. In a preferred embodiment of the present invention, when detecting abnormal operation of the human-body information sensing device 10, the abnormity sensing unit 10D first stops the human-body information sensing device 10, and restarts the human-body information sensing device 10 when the human-body information sensing device 10 recovers its normality. For example, the abnormity sensing unit 10D detects operation of the human-body information sensing device 10 by sensing abnormities in terms of temperature, voltage, and/or current (not limiting).

As shown in FIG. 1 through FIG. 3, in the embodiment of the present invention, the human-body information sensing device 10 comprises a temperature sensing unit 10E and a protecting unit 10F that are coupled to the first wireless communication unit 10A. The temperature sensing unit 10E senses an intra-body temperature value inside the human body. The protecting unit 10F is preprogrammed with a temperature standard value and a time threshold. When the intra-body temperature value stays higher than the temperature standard value for a time period exceeding the time threshold, the protecting unit 10F controls the human-body information sensing device 10 to stop operating. For example, assuming that the temperature standard value is 36 °C and the time threshold is 1 minute, when the intra-body temperature value has become 42 °C (i.e., being higher than the temperature standard value), and has stayed at this level for a period longer than 1 minute (being higher than the time threshold), the protecting unit 10F shuts down all components in the human-body information sensing device 10 except for those vital (such as the temperature sensing unit 10E), so as to protect the human-body information sensing device 10 from damage.

At this time, since the temperature sensing unit 10E remains working and continuously senses the intra-body temperature value, when it detects that the intra-body temperature value has become lower than the temperature standard value, the protecting unit 10F can restart the shut-off components in the human-body information sensing device 10, so as to restore the full ability of the human-body information sensing device 10 to collect various data in the human body.

As shown in FIG. 1 through FIG. 3, in the embodiment of the present invention, the human-body information sensing device 10 comprises a pH value sensing unit 10G that is coupled to the first wireless communication unit 10A. The pH value sensing unit 10G senses an intra-body pH value inside the human body, and outputs the intra-body pH value to the off-body processing device 20 through the first wireless communication unit 10A. Thus, the off-body processing device 20 receives the intra-body absolute pressure value and the intra-body pH value and determines a location information of the human-body information sensing device 10 accordingly so that the user can tell where the received data are collected in the human body.

As shown in FIG. 1 through FIG. 3, in the embodiment of the present invention, the human-body information sensing device 10 comprises an internal movement sensing unit 10H that is coupled to the first wireless communication unit 10A. The internal movement sensing unit 10H senses an internal human-body movement information related to the human body. The first wireless communication unit 10A sends the internal human-body movement information to the off-body processing device 20, so that the off-body processing device 20 can assess intra-body gastrointestinal peristalsis in the human body according to the internal human-body movement information. In the embodiment of the present invention, the internal movement sensing unit 10H is an inertial measurement unit (IMU). The internal human-body movement information measured by the internal movement sensing unit 10H, such as the acceleration, the angular acceleration, etc., is data having periodic curve variations. With this information, the off-body processing device 20 can assess intra-body gastrointestinal peristalsis in the human body more accurately.

As shown in FIG. 1 through FIG. 3, in the embodiment of the present invention, the human-body information sensing device 10 comprises a first counter 10I that is coupled to the first wireless communication unit 10A. The first counter 10I records a first time stamp of the intra-body absolute pressure value collected by the first pressure sensing unit 10B, and outputs the first time stamp to the off-body processing device 20 through the first wireless communication unit 10A for the off-body processing device 20 to process subsequently.

The off-body processing device 20 is in communication with the human-body information sensing device 10. The off-body processing device 20 has a second wireless communication unit 20A, a second pressure sensing unit 20B, and a pressure calculating unit 20C. The second wireless communication unit 20A is in communication with the first wireless communication unit 10A, so as to receive the various data and information sent from the human-body information sensing device 10 through the first wireless communication unit 10A. The second pressure sensing unit 20B senses an ambient absolute pressure value. The pressure calculating unit 20C receives the intra-body absolute pressure value and the ambient absolute pressure value, and determines an intra-body relative pressure value by calculating the difference between the intra-body absolute pressure value and the ambient absolute pressure value. Thus, the medical staff is enabled to have a clear picture of the health of the user (i.e., a patient) from the intra-body relative pressure value and plan proper treatment accordingly.

As shown in FIG. 1 through FIG. 3, in the embodiment of the present invention, the off-body processing device 20 comprises a pressure alarm unit 20D and a display unit 20E coupled to the second wireless communication unit 20A. The pressure alarm unit 20D is preprogrammed with a relative pressure standard range. The pressure alarm unit 20D compares the intra-body relative pressure value and the relative pressure standard range. If the intra-body relative pressure value goes beyond the relative pressure standard range, the pressure alarm unit 20D issues a pressure alarm signal. The display unit 20E receives the pressure alarm signal and displays a relative pressure abnormity information accordingly for the user or the medical staff taking care of the user to check.

Therein, as shown in FIG. 1 through FIG. 3, in the embodiment of the present invention, when the abnormity sensing unit 10D detects abnormal operation, the display unit 20E receives the operational abnormity signal through the first wireless communication unit 10A and the second wireless communication unit 20A, and displays an operational abnormity message according to the operational abnormity signal to inform the user or the medical staff taking care of the user of the abnormity of the human-body information sensing device 10.

As shown in FIG. 1 through FIG. 3, in the embodiment of the present invention, the off-body processing device 20 comprises a remote control unit 20F that is coupled to the second wireless communication unit 20A. The remote control unit 20F outputs an activating signal to the activating unit 10F through the first wireless communication unit 10A and the second wireless communication unit 20A. The activating unit 10F then in response to the activating signal controls the human-body information sensing device 10 to start to operate. Since the activating unit 10F of the human-body information sensing device 10 is a non-contact switch, when the human-body information sensing device 10 is inside the human body, the user can use the remote control unit 20F to control the human-body information sensing device 10 to operate, making use of the disclosed system more versatile and convenient.

As shown in FIG. 1 through FIG. 3, in the embodiment of the present invention, off-body processing device 20 comprises a location analyzing unit 20G that is coupled to the second wireless communication unit 20A. The location analyzing unit 20G receives the intra-body relative pressure value and the intra-body pH value through the second wireless communication unit 20A and obtains a location information related to the human-body information sensing device 10 according to the intra-body relative pressure value and the intra-body pH value, for the user or the medical staff to know where the human-body information sensing device 10 is, and know to which body part the data displayed in the display unit 20E are related. Therein, the location analyzing unit 20G determines the location information according to variations of the intra-body relative pressure value and the intra-body pH value.

As shown in FIG. 1 through FIG. 3, in the embodiment of the present invention, the off-body processing device 20 comprises an external movement sensing unit 20H and a movement state calculating unit 20I that are coupled to the second wireless communication unit 20A. The external movement sensing unit 20H senses an external movement information of the human-body. The movement state calculating unit 20I receives the internal human-body movement information through the second wireless communication unit 20A, and calculates an actual gastrointestinal peristalsis information according to the internal human-body movement information and the external movement information, so that the user can be informed of how good his/her gastrointestinal peristalsis is through off-body processing device 20. Therein, the actual gastrointestinal peristalsis information comprises the internal human-body movement information, the external movement information, and an actual gastrointestinal pressure waveform variation information, so that the user can assess how good his/her gastrointestinal peristalsis is according to variations of the waveform more clearly and easily.

As shown in FIG. 1 through FIG. 3, in the embodiment of the present invention, off-body processing device 20 comprises a second counter 20J and a synchronization unit 20K that are coupled to the second wireless communication unit 20A. The second counter 20J records a second time stamp of the ambient absolute pressure value sensed by the second pressure sensing unit 20B. The synchronization unit 20K receives the first time stamp and the second time stamp through the second wireless communication unit 20A, and synchronizes the pressure sensing timing between the first pressure sensing unit 10B and the second pressure sensing unit 20B according to the first time stamp and the second time stamp, so as to allow the pressure calculating unit 20C to correctly receive the intra-body absolute pressure value and the ambient absolute pressure value of the same pressure sensing timing, thereby correctly determining the intra-body relative pressure value.

As shown in FIG. 1 through FIG. 3, in the embodiment of the present invention, the off-body processing device 20 comprises a storing unit 20L that is coupled to the second wireless communication unit 20A. The storing unit 20L receives and stores various data and information sensed and figured out by the human-body information sensing device 10 and the off-body processing device 20, so that a developer is enabled to optimize and improve the disclosed system according to the data stored in the storing unit 20L.

As shown in FIG. 1 through FIG. 3, in the embodiment of the present invention, the off-body processing device 20 comprises a remote communication unit 20M that is coupled to the storing unit 20L. The remote communication unit 20M is remotely connected to a cloud server 200. The remote communication unit 20M transmits the various data stored in the storing unit 20L to the cloud server 200 for better storage and protection of the data. This is helpful in reducing risks of data loss and allows the developer to collect data remotely through the cloud server 200 for analysis, which enables optimization and improvement of the system.

As shown in FIG. 1 through FIG. 3, in the embodiment of the present invention, the off-body processing device 20 comprises a recording unit 20N that is coupled to the second wireless communication unit 20A. When the user feels unwell, he/she can use the recording unit 20N to record unwell time data. Medical staff can later, according to the unwell time data, review variations of vital signs happening when the user (i.e., a patient) felt unwell, thereby finding out possible reasons of the unwell feeling of the user and wisely making therapeutic strategies for the user. Therein, the recording unit 20N may be a virtual button displayed through the display unit 20E or a physical button installed at the edge of the off-body processing device 20, so that when the user feels unwell, he/she can activate the recording unit 20N promptly.

Thereby, the human-body information sensing device 10 and the off-body processing device 20 of the present invention can accurately detect the intra-body absolute pressure value and the intra-body relative pressure value inside the human body. With this, the present invention provides medical staff with accurate and diverse data, thereby achieving competent monitoring of vital signs of patients.

Moreover, the human-body information sensing device 10 of the present invention further has additional components for detecting various data related to the human body (such as the intra-body pH value, the gastrointestinal peristalsis state, etc.). These additional components may include those configured to monitor operation of various components of the device. These data are uploaded to the off-body processing device 20, so that the medical staff can ensure proper operation of the device all the time, thereby preventing failure in accurate monitoring of the patient's vital signs caused by breakdown of any of the components.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A human-body pressure sensing system, comprising:
a human-body information sensing device, having a first wireless communication unit, a first pressure sensing unit, and an activating unit, in which the first pressure sensing unit senses an intra-body absolute pressure value inside a human body, and outputs the intra-body absolute pressure value through the first wireless communication unit, so as that the activating unit controls the human-body information sensing device to start operation; and
an off-body processing device, having a second wireless communication unit, a second pressure sensing unit, and a pressure calculating unit, in which the second wireless communication unit is in communication with the first wireless communication unit, and the second pressure sensing unit senses an ambient absolute pressure value, so that the pressure calculating unit receives the intra-body absolute pressure value and the ambient absolute pressure value, and determines an intra-body relative pressure value by calculating a difference between the intra-body absolute pressure value and the ambient absolute pressure value.

2. The human-body pressure sensing system of claim 1, wherein the off-body processing device comprises a pressure alarm unit and a display unit that are coupled to the second wireless communication unit, in which the pressure alarm unit is preprogrammed with a relative pressure standard range, and the pressure alarm unit compares the intra-body relative pressure value and the relative pressure standard range, so that if the intra-body relative pressure value goes beyond the relative pressure standard range, the pressure alarm unit issues a pressure alarm signal to the display unit, and the display unit displays a relative pressure abnormity information according to the pressure alarm signal.

3. The human-body pressure sensing system of claim 2, wherein the human-body information sensing device comprises an abnormity sensing unit that is coupled to the first wireless communication unit, in which the abnormity sensing unit senses the operation of the human-body information sensing device, and when sensing any abnormity related to the operation, the abnormity sensing unit outputs an operational abnormity signal to the display unit through the first wireless communication unit and the second wireless communication unit, so that the display unit displays an operational abnormity message according to the operational abnormity signal.

4. The human-body pressure sensing system of claim 1, wherein the human-body information sensing device comprises a temperature sensing unit and a protecting unit, in which the temperature sensing unit senses an intra-body temperature value inside the human body, and the protecting unit is preprogrammed with a temperature standard value and a time threshold, so that when the intra-body temperature value has stayed at a level higher than the temperature standard value for a time period longer than the time threshold, the protecting unit controls the human-body information sensing device to stop operating.

5. The human-body pressure sensing system of claim 1, wherein the off-body processing device comprises a remote control unit that is coupled to the second wireless communication unit, in which the remote control unit outputs an activating signal to the activating unit through the first wireless communication unit and the second wireless communication unit, so that the activating unit controls the human-body information sensing device to start the operation according to the activating signal.

6. The human-body pressure sensing system of claim 1, wherein the human-body information sensing device comprises a pH value sensing unit that is coupled to the first wireless communication unit, in which the pH value sensing unit senses an intra-body pH value inside the human body, and outputs the intra-body pH value through the first wireless communication unit; and wherein the off-body processing device comprises a location analyzing unit that is coupled to the second wireless communication unit, in which the location analyzing unit receives the intra-body relative pressure value and the intra-body pH value through the second wireless communication unit, and obtains a location information of the human-body information sensing device according to the intra-body relative pressure value and the intra-body pH value.

7. The human-body pressure sensing system of claim 1, wherein the human-body information sensing device comprises an internal movement sensing unit, in which the internal movement sensing unit senses an internal human-body movement information related to the human body; and wherein the off-body processing device comprises an external movement sensing unit, in which the external movement sensing unit senses an external movement information related to the human body.

8. The human-body pressure sensing system of claim 7, wherein the off-body processing device comprises a movement state calculating unit, which is coupled to the second wireless communication unit and the external movement sensing unit, in which the movement state calculating unit receives the internal human-body movement information through the second wireless communication unit, and determines an actual gastrointestinal peristalsis information by performing calculation according to the internal human-body movement information and the external movement information.

9. The human-body pressure sensing system of claim 8, wherein the actual gastrointestinal peristalsis information comprises the internal human-body movement information, the external movement information, and an actual gastrointestinal pressure waveform variation information.

10. The human-body pressure sensing system of claim 1, wherein the off-body processing device comprises a recording unit that is coupled to the second wireless communication unit, so that when feeling unwell, a user is allowed to record an unwell time data using the recording unit.
